(19) United States
(12) Patent Application Publication
(10) Pub. No.: US 2026/0119574 A1
(43) Pub. Date: Apr. 30, 2026

(54) WORD EMBEDDINGS CONTEXTUALIZED FOR A SPECIALIZED DOMAIN SUCH AS MEDICINE

(71) Applicant: Providence St. Joseph Health, Renton, WA (US)

(72) Inventors: M.V. Udai Shankar, Hyderabad (IN); Sri Pallavi M A, Hyderabad (IN); Cibe S Kumaresan, Virudhunagar (IN); Lalit Dhakar, Hyderabad (IN); Kiran Pilli, Hyderabad (IN); Lagnesh T S, Tiruchirappalli (IN); Anudeep S Appe, Hyderabad (IN); Preetha Kumar, Hyderabad (IN); Hari Atmakuri, Hyderabad (IN); Preeti Nair, Hyderabad (IN); Krishna K Matham, Hyderabad (IN)

(21) Appl. No.: 18/933,521

(22) Filed: Oct. 31, 2024

(51) Int. Cl. G06F 16/901

(52) U.S. Cl. CPC G06F 16/9024

(57) ABSTRACT

A text vector facility is described that establishes a graph data structure for a specialized domain. The graph includes a number of first nodes, each identifying a term relevant to the specialized domain, and specifying a vector representing a meaning of the identified term. The graph also includes a number of second nodes, each identifying a term relevant to the distinguished specialized domain and not initially specifying any vector representing a meaning of the identified term. The graph further includes, for each of the second nodes, one or more edges each connecting the second node to one of the first nodes. The graph is usable to perform graph learning to establish, for each of the second nodes, a vector representing a meaning of the term identified by the second node.

### BACKGROUND

Language models such as large language models, small language models, and transformers operate on text. Much of the work that these language models do is reliant on the meaning of words, word portions, and words groups, referred to herein as “terms.”

In order to facilitate this work on text that relies on the meaning of the terms it contains, a common early step in applying these language models is transforming terms into representations of their meaning whose level of relatedness can be quantitively assessed. In many cases, these representations are vectors each specifying a particular position in a multidimensional embedding space. The level of similarity of such vectors—and therefore the level of similarity of the terms they represent—can be determined using similarity measures such as the cosine similarity measure, which determines the cosine of an angle defined by two vectors.

### BRIEF DESCRIPTION OF THE DRAWINGS

FIG. 1 is a block diagram showing some of the components typically incorporated in at least some of the computer systems and other devices on which the facility operates.

FIG. 2 is a flow diagram showing a process performed by the facility in some embodiments to make use of a vector set for a specialized domain vocabulary.

FIGS. 3-5 are graph diagrams showing sample graphs depicting an example of establishing a vector set for a specialized domain vocabulary.

FIG. 6 is a flow diagram showing a process performed by the facility in some embodiments to establish a vector set for a specialized domain vocabulary.

FIG. 7 is a graph diagram showing a sample graph to which the facility has added nodes based upon the prescription code records shown in Table 1.

FIG. 8 is a graph diagram showing a sample graph to which the facility has added nodes for the portion of the medical diagnosis code hierarchy shown in Table 2.

### DETAILED DESCRIPTION

The inventors have recognized that using existing embedding schemes or models—which are created based on wide-ranging general-purpose corpora—to determine vector representations for terms used in a specialized domain such as medicine can have significant disadvantages. Reasons include that some terms in the specialized domain may be unique to the specialized domain, and not well represented in the general-purpose corpus used to construct the existing embedding scheme or model, such that no vector is available to assign to some terms commonly used in the specialized domain. Additionally, terms as used in the specialized domain may have meanings there that diverge from their meaning in the general-purpose corpus, causing vectors to be assigned to some terms commonly used in the specialized domain that represent a meaning not consistent with these terms' meanings in the specialized domain.

In response to recognizing these disadvantages of using conventional embedding schemes or models to generate embedding vectors for text from a specialized domain, the inventors have conceived and reduced to practice a software and/or hardware facility for word embeddings contextualized for a specialized domain such as medicine. In particular, the facility uses graph learning techniques to bootstrap embedding vectors available for some words in the domain in a particular embedding space to embedding vectors for other words in the domain that are in the same embedding space, such that the vectors consistently represent the meaning of words across the domain.

The facility begins with a collection of word vectors that is effective in expressing the meaning of terms occurring in the specialized domain's vocabulary. For example, where the specialized domain is medicine, the facility begins with an open-source set of word vectors that include medical terms, such as one or both of the word vector sets introduced in (a) Zhang Y, Chen Q, Yang Z, Lin H, Lu Z, BioWordVec, improving biomedical word embeddings with subword information and MeSH, Scientific Data. 2019, available at www.nature.com/articles/s41597-019-0055-0; and (b) Chen Q, Peng Y, Lu Z. BioSentVec: creating sentence embeddings for biomedical texts, The 7th IEEE International Conference on Healthcare Informatics, 2019, available at arxiv.org/abs/1810.09302, each of which is hereby incorporated by reference in its entirety. Where a document incorporated herein by reference conflicts with the present application, the present application controls. These sets of word vectors are available at github.com/ncbi-nlp/BioSentVec.

In some embodiments, the facility filters its beginning collection of word vectors to those representing terms in a list of terms that is more effectively focused on the specialized domain's vocabulary. For example, where the specialized domain is medicine, the facility filters its beginning collection of word vectors to those vectors representing medical terms represented in a separate set of word vectors for this domain, such as the set of word vectors included with a spaCy model, such as the medium spaCy model available at spacy.io/models/en #en_core_web_md, or the large spaCy model available at https://spacy.io/models/en #en_core_web_Ig. In some embodiments, the facility further or instead reduces the beginning collection of word vectors to collapse groups of vectors each corresponding to variable capitalizations of the same term.

Following any filtering and other reduction of the beginning collection of word vectors, the facility constructs a graph neural network (“GNN”), such as a graph convolutional network (“GCN”) in which each node is one of these word vectors and the term it defines. In some embodiments, the edges between pairs of nodes in this graph are labeled with a weight representing the level of similarity between the terms represented by the nodes of the pair, obtained by determining a similarity measure between the vectors of the two nodes. Thus, an edge between two nodes whose vectors are very similar has a large weight, whereas an edge between two nodes whose vectors are dissimilar has a small weight.

To expand the word vector set represented by this graph, the facility (a) adds to the graph a number of new nodes representing terms of the specialized domain's vocabulary that are in a particular category or are from a particular source, and (b) uses graph learning techniques to learn vectors for these new nodes that are in the same embedding space as the vectors in the graph's initial nodes. For example, where the specialized domain is medicine, in various embodiments the facility performs this process with one or more of the following categories of terms: (1) medical diagnosis codes, such as ICD diagnosis codes defined at icd.who.int/en; (2) medical procedure codes, such as Current Procedural Terminology (“CPT”) procedure codes defined at www.cms.gov/medicare/regulations-guidance/physician-self-referral/list-cpt-hcpcs-codes; (3) prescription codes, such as RxNorm prescription codes defined at www.nlm.nih.gov/research/umls/rxnorm/index.html; and (4) lab result codes, such as Logical Observation Identifiers Names and Codes (“LOINC”) lab result codes defined at loinc.org.

A source of a group of terms used to create new nodes typically describes that term with reference to other terms. For example, a source of prescription codes typically provides attributes for a prescription code, such as indication (e.g., for a medicine called “Adthyza Pill” having prescription code “2671589,” indication is “hypertension”), medicine type (e.g., “antihypertensive”), and side effect (e.g., “dizziness”). When adding nodes to the graph for a category of terms, the facility creates edges between each new node and existing nodes for some or all of the terms used by the source to describe the term of the new node. To continue the example above, when adding a node to the graph for the medicine “Adthyza Pill,” the facility connects this new node to nodes for “2671589,” “hypertension,” “antihypertensive,” and “dizziness.” In some embodiments, the facility labels these edges with the attribute whose value is represented by the existing connected node.

In some embodiments, where a category of terms is organized in a hierarchy, the facility uses adjacency in the hierarchy to establish additional edges between nodes added for terms of this category. For example, where diagnosis codes are defined hierarchically, in some embodiments the facility adds an edge between the new node for a diagnosis code and a new node for that diagnosis code's parent in the hierarchy.

Once the facility has added new nodes to the graph in some or all of the ways described above, it applies GNN learning techniques to the graph in order to learn vectors for the new nodes that are in the same embedding space as the original nodes. For example, in some embodiments, the facility uses the technique described in Yu, D., Yang, Y., Zhang, R. and Wu, Y., 2021 April, Knowledge embedding based graph convolutional network, In Proceedings of the web conference 2021 (pp. 1619-1628), available at dl.acm.org/doi/10.1145/3442381.3449925, which is hereby incorporated by reference in its entirety.

At the completion of the learning process, the vectors contained by the graph for all of the nodes represent the meaning of all of the terms corresponding to the nodes in the same embedding space. This set of vectors can be used to perform deep learning tasks against documents in the specialized domain. For example, the set of vectors can be used to create representations of patient records in an electronic medical record (“EMR”) system that are usable to learn to make predictions about a patient from the contents of the patient's EMR record.

By operating in some or all of the ways described above, the facility generates rich and internally-consistent meaning representations of the vocabulary for a specialized domain such as medicine.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, deep learning tasks can be performed against representations of documents in the specialized domain that use a vector set determined by the facility using lower levels of processing resources based on the consistency of meaning established in the vector set by the facility.

Further, for at least some of the domains and scenarios discussed herein, the processes described herein as being performed automatically by a computing system cannot practically be performed in the human mind, for reasons that include that the starting data, intermediate state(s), and ending data are too voluminous and/or poorly organized for human access and processing, and/or are a form not perceivable and/or expressible by the human mind; the involved data manipulation operations and/or subprocesses are too complex, and/or too different from typical human mental operations; required response times are too short to be satisfied by human performance; etc. As one example, the human mind cannot practically manage nor perform the vast volume of computations required to implement the neural network learning processes used by the facility in some embodiments.

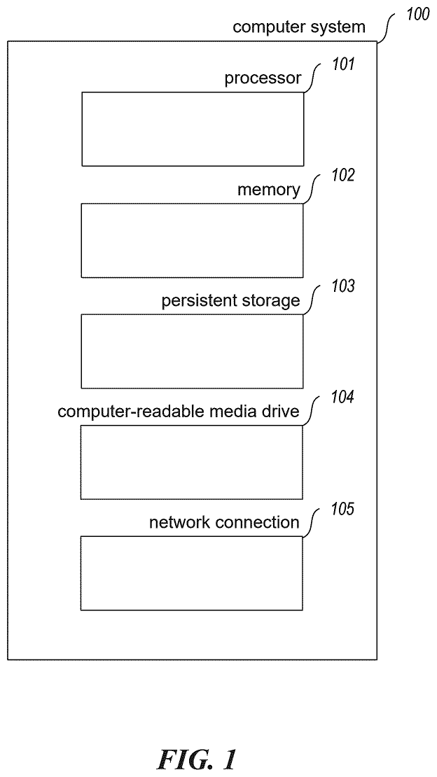

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102—such as RAM, SDRAM, ROM, PROM, etc.—for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. None of the components shown in FIG. 1 and discussed above constitutes a data signal per se. While computer systems configured as described above are typically used to support the operation of the facility, those skilled in the art will appreciate that the facility may be implemented using devices of various types and configurations, and having various components.

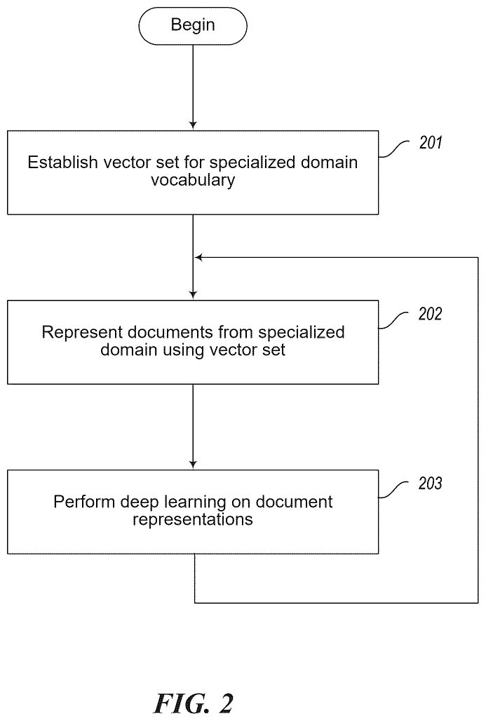

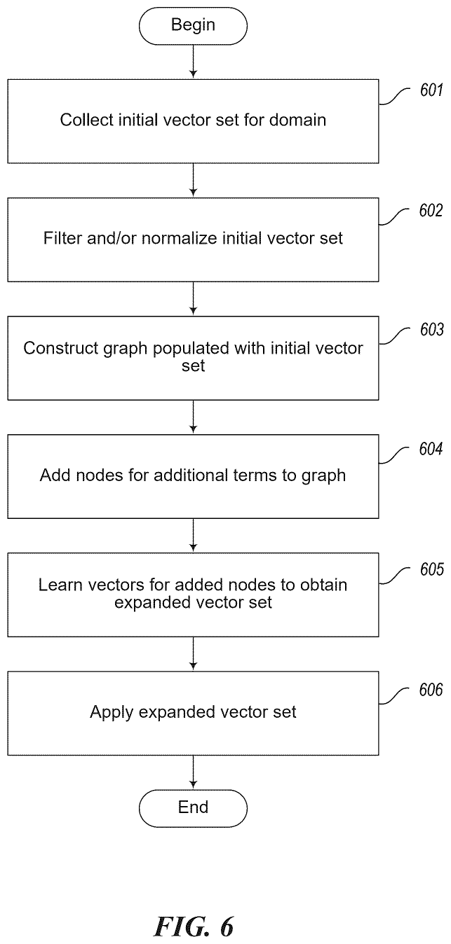

FIG. 2 is a flow diagram showing a process performed by the facility in some embodiments to make use of a vector set for a specialized domain vocabulary. In act 201, the facility establishes a vector set for the specialized domain vocabulary. Details of act 201 are discussed in connection with FIG. 6 below. In act 202, the facility represents one or more documents from the specialized domain using the vector set established in act 201, such as by replacing some or all of the textual terms in each document with the corresponding vectors in the vector set. In act 203, the facility performs deep learning on the document representations obtained in act 202. In various embodiments, act 203 involves training a deep learning model using the document representations, and/or applying a trained deep learning to the document representations. After act 203, the facility continues in act 202 to process additional documents.

Those skilled in the art will appreciate that the acts shown in FIG. 2 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

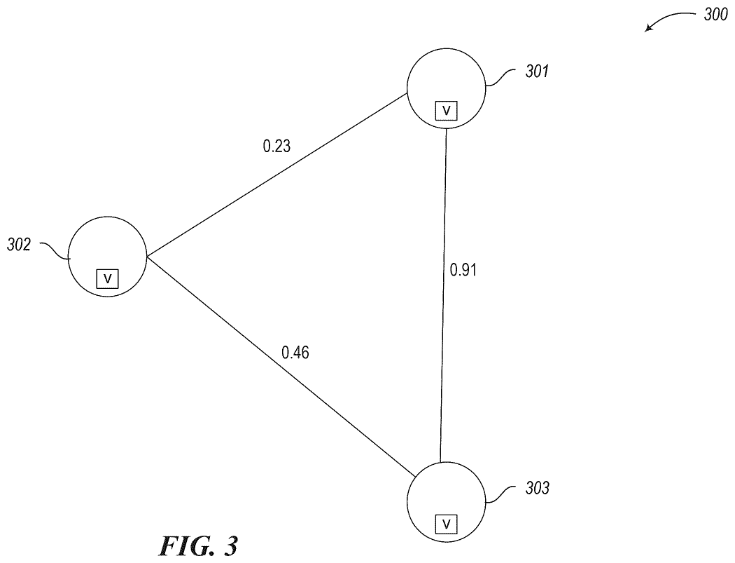

FIGS. 3-5 are graph diagrams showing sample graphs depicting an example of establishing a vector set for a specialized domain vocabulary. FIG. 3 is an initial graph diagram showing a graph 300 having nodes 301-303. Each node corresponds to a particular term in the specialized vocabulary, and contains a vector representing the meaning of the word. (The presence of the vector is shown by the letter “V” in a box inside the node). Edges between each pair of nodes show a similarity measure between the vectors of those nodes. In this case, nodes 301-303 represent terms of the vocabulary that have vectors in the initial vector set.

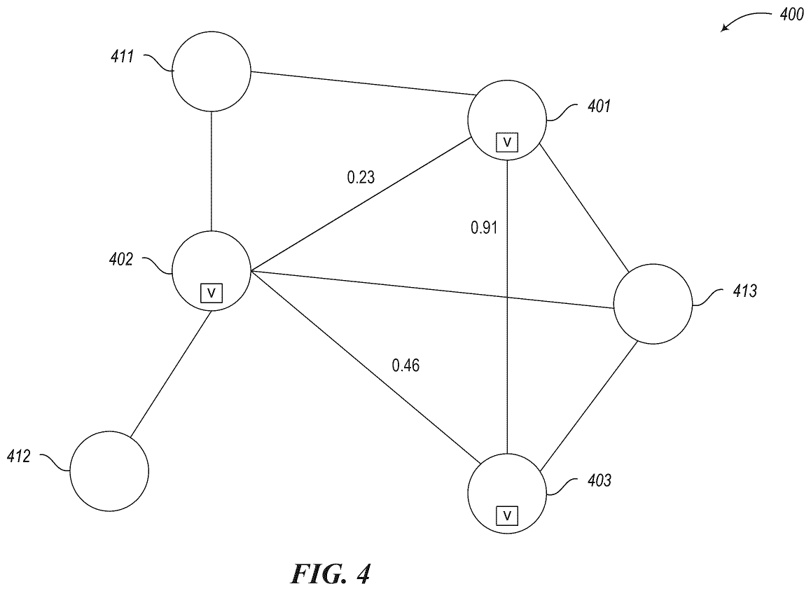

FIG. 4 shows a second graph 400 in which nodes 411-413 have been added, each corresponding to a term for which the initial vector set does not contain a vector. The absence of a “V” in these nodes indicate that no vector is at this point contained by these nodes.

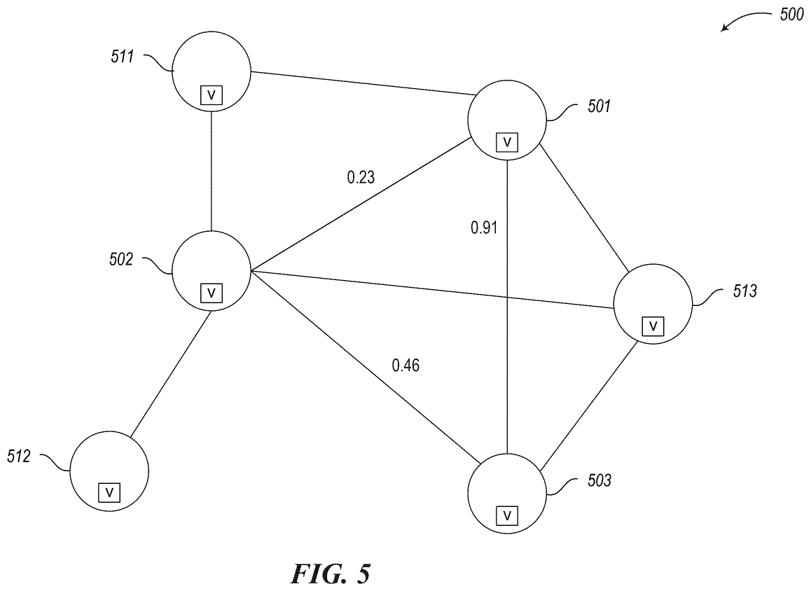

FIG. 5 shows a third sample graph 500, in which graph learning has been used to learn vectors for added nodes 511-513. These learned vectors are in the same embedding space as the vectors from the initial vector set.

FIG. 6 is a flow diagram showing a process performed by the facility in some embodiments to establish a vector set for a specialized domain vocabulary. In act 601, the facility collects an initial vector set for the specialized domain, such as the BioWordVec or BioSentVec vector sets. In act 602, the facility filters and/or normalizes the initial vector set collected in act 601. In some embodiments, the facility performs filtering by removing vectors from the vector set for terms that are not in a list of terms for the domain, such as the list of words for the medical domain incorporated in one or more of the spaCy models. In some embodiments, the facility normalizes the initial vector set by collapsing each group of terms amounting to a different form of capitalization of the same term to a single vector, such as by averaging the vectors in each group for each term.

In act 603, the facility constructs a graph in which each node represents a vector from the initial vector set, as adjusted in act 602, accompanied by the vector's term. In some embodiments, the constructed graph is a GCN or other GNN. In some embodiments, the facility establishes in the graph edges between the nodes labeled with a level of similarity between the vectors of each pair of nodes, such as by using the cosine similarity measure, a spatial distance vector similarity measure, etc.

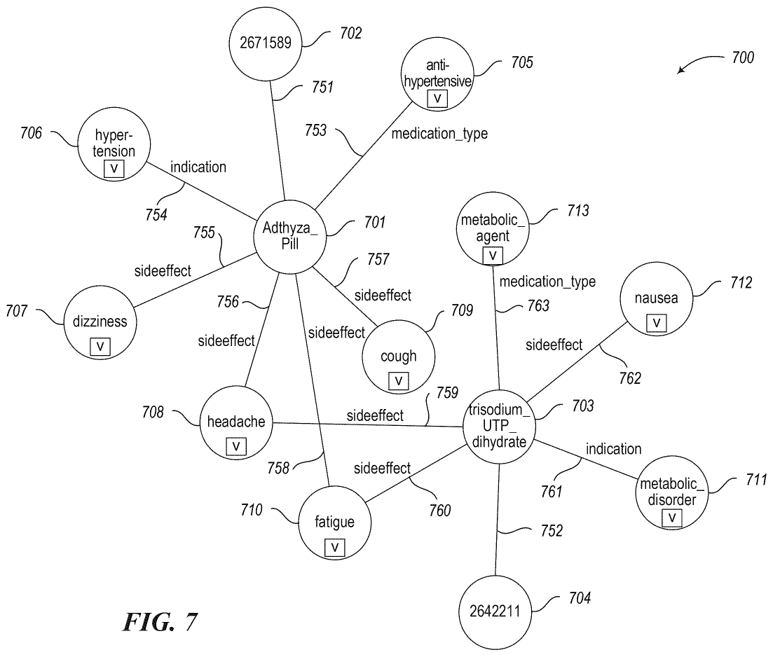

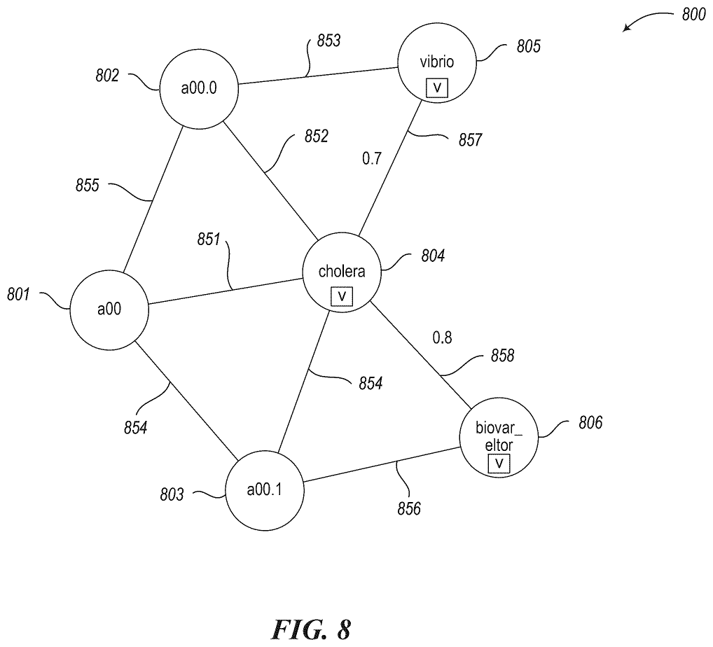

In act 604, the facility adds additional nodes to the graph constructed in act 603 for additional terms significant to the domain's vocabulary. Where the domain is medicine, these additional terms can be from categories such as medical diagnosis codes, medical procedures codes, prescription codes, and lab results codes. Examples of the facility's addition of nodes for sample categories of additional terms is discussed below in connection with FIGS. 7 and 8. In act 605, the facility uses graph learning techniques to learn vectors for the nodes added to the graph in act 604. After performing this learning, the vectors represented by all the nodes in the graph constitute an expanded vector set. In act 606, the facility applies the expanded vector set, such as to transform text relating to the specialized domain into a representation of that text's meaning. After act 606, this process concludes.

In some embodiments, the facility adds nodes to its graph to represent prescription codes, such as RxNorm prescription codes. Table 1 below contains information about the RxNorm prescription codes for two medicines, “Adthyza Pill” and “trisodium UTP dihydrate.”

TABLE 1
RXNORM PRESCRIPTION CODES

| | |
|---|---|
| 1 | ‘Adthyza Pill’: { |
| 2 | ‘rxnorm’: 2671589 |
| 3 | ‘indication’: [‘hypertension’], |
| 4 | ‘type of medicine’: [‘antihypertensive’], |
| 5 | ‘dosage range’: (1, 5), |
| 6 | ‘dosage unit’: ‘mg’, |
| 7 | ‘side effects’: [‘dizziness’, ‘headache’, ‘fatigue’, ‘cough’] |
| 8 | } |
| 9 | ‘trisodium UTP dihydrate’: |
| 10 | { |
| 11 | ‘rxnorm’: 2642211 |
| 12 | ‘indication’: [‘metabolic disorders’], |
| 13 | ‘type of medicine’: [‘metabolic agent’], |
| 14 | ‘dosage range’: (0, 1000), |
| 15 | ‘dosage unit’: ‘mg’, |
| 16 | ‘side effects’: [‘nausea’, ‘vomiting’, ‘headache’, ‘fatigue’], |
| 17 | } |

Table 1 contains two RxNorm records: a first for the medicine “Adthyza Pill” in lines 1-8, and a second for the medicine “trisodium UTP dihydrate” in lines 9-17. In each case, the record provides values for the following attributes of the medicine: name (e.g., in line 1); prescription code (e.g., in line 2); indication that the administration of the medicine is proper (e.g., in line 3); medicine type (e.g., in line 4); a proper dosage range and its units (e.g., lines 5 and 6, respectively); and side effects that are possible with the medicine's use (e.g., line 7).

FIG. 7 is a graph diagram showing a sample graph to which the facility has added nodes based upon the prescription code records shown in Table 1. Those skilled in the art will appreciate that in practice the graph 700 may contain a much larger number of nodes; to make FIG. 7 more intelligible, only nodes of the graph most immediately relevant to the added nodes are shown. The nodes in graph 800 shown in FIG. 8 and discussed below is similarly shown with a limited set of the graph's nodes.

For the first prescription code record, the facility has added nodes 701—for the medicine name—and 702—for the medicine's prescription code. These nodes are connected by edge 751. Node 701 for the first medicine's name is also connected via edges to original nodes of the graph corresponding to terms used or implicated in the prescription code record: node 705 for the term “anti-hypertensive,” connected to node 701 by edge 753 labelled to indicate that this term is the medicine's type; node 706 for the term “hypertension,” connected to node 701 by edge 754 indicating that this term is the medicine's indication; node 707 for the term “dizziness,” connected to node 701 by edge 755 indicating that this term is the medicine's side effect; node 708 for the term “headache,” connected to node 701 by edge 756 indicating that this term is a side effect of the medicine; node 709 for the term “cough,” connected to node 701 by edge 757 indicating that this term is a side effect of the medicine; and node 710 for the term “fatigue,” which is connected to node 701 by edge 758 indicating that this term is a side effect of the medicine. The facility similarly connects added nodes 703 and 704 for the medicine described in the second prescription code record shown in Table 1. The facility applies graph learning techniques to learn vectors for added nodes 701-704 in the same embedding space in which the other vectors in the graph represent the meaning of the corresponding terms.

In some embodiments, the facility adds nodes to its graph to represent medical diagnosis codes, such as ICD medical diagnosis codes. Table 2 below shows a portion of a medical diagnosis code hierarchy directed to the disease cholera.

TABLE 2
ICD A00 MEDICAL DIAGNOSIS CODE HIERARCHY

| | |
|---|---|
| 1 | A00 Cholera |
| 2 | A00.0 Cholera due to Vibrio cholerae 01, biovar cholerae |
| 3 | A00.1 Cholera due to Vibrio cholerae 01, biovar eltor |

It can be seen that the portion of the medical diagnosis code hierarchy shown in Table 2 includes 3 codes: in line 1 a general code for cholera: in line 2, a code for a first variant of cholera; and in line 3 a code for a second variant of cholera. Based upon their organization of characters and the levels of indentation at which they are shown, cholera variant codes A00.0 and A00.1 are each established as children of the more general cholera code A00.

FIG. 8 is a graph diagram showing a sample graph to which the facility has added nodes for the portion of the medical diagnosis code hierarchy shown in Table 2. In graph 800, the facility has added node 801 for the general cholera code A00, and nodes 802 and 803 for the cholera variant codes A00.0 and A00.1, respectively. Because each of the cholera variant codes are children of the general cholera code, new nodes 802 and 803 are each connected to node 801, via edges 855 and 854, respectively. It can be seen that each of the added nodes is connected by edges to one or more initial nodes of the graph having vectors. For example, node 803 corresponding to line 3 of Table 2, is connected to node 804 containing a vector for the term “cholera” that occurs in the description of code A00.1, as well as to node 806 that contains a vector for the term “biovar eltor” also present in the description of this diagnosis code. From this point, the facility uses graph learning techniques to learn vectors for added nodes 801-803.

It is common for graph known learning techniques to rely on a loss function that mathematically and/or logically expresses one or more goals for the learning. In the realm of graph neural networks, specifically in the context of a Graph Convolutional Network (GCN), node embeddings play an important role in capturing the structural and feature-based relationships between nodes. The contrastive_loss function used by the facility in some embodiments is a basis for refining these embeddings to ensure they accurately reflect the graph's underlying connectivity while maintaining stability relative to their initial representations.

The contrastive_loss function is designed to achieve three main goals: enhance Similarity of Connected Nodes (Positive Loss); Increase Similarity of Unconnected Nodes (Negative Loss); and Regularize to Maintain Original Embeddings (Regularization Loss).

The primary objective of the Positive Loss function is to ensure that nodes directly connected by edges in the graph have similar embeddings. To achieve this, it creates a positive mask to identify which pairs of nodes are connected. Using this mask, the function computes the pairwise cosine similarities between embeddings of all nodes. For pairs identified as positive (connected), it calculates the positive loss, which measures the difference between their current cosine similarity and a predefined margin. The goal here is to minimize this difference, effectively making the embeddings of connected nodes more similar to each other.

In contrast to the positive loss, the Negative Loss function also aims to ensure that nodes not connected by edges have dissimilar embeddings. This is achieved by calculating the negative loss, which is derived from the cosine similarity of node pairs that are not directly connected. The function encourages these similarities to be high, promoting the separation of embeddings for non-adjacent nodes. By doing so, it helps in distinguishing between nodes that should be perceived as different based on their non-adjacency in the graph.

To prevent drastic changes to the embeddings from their initial values, the Regularization Loss function includes a regularization term. This term penalizes large deviations of the current embeddings from their initial representations. Specifically, it calculates the similarity between the normalized current embeddings and the normalized initial embeddings. The regularization loss is then added to the overall loss function, scaled by a regularization parameter, lambda. This ensures that while the embeddings are refined to capture graph connectivity more accurately, they do not stray too far from their original values, maintaining stability and consistency.

In some embodiments, the facility uses a loss function such as the loss function shown below in Table 3.

TABLE 3
GRAPH CONVOLUTION NETWORK LOSS FUNCTION

| | |
|---|---|
| 1 | def contrastive_loss(embeddings, edge_index, |
| 2 | initial_embeddings, |
| 3 | device=device, margin=1.0, |
| 4 | lambda_reg=0.2): |
| 5 | # Create a positive mask to identify positive pairs |
| 6 | pos_mask = torch.zeros((embeddings.size(0), \ |
| 7 | embeddings.size(0)), \ |
| 8 | dtype=torch.bool, device=device) |
| 9 | pos_mask[edge_index[0], edge_index[1]] = True |
| 10 | pos_mask[edge_index[1], edge_index[0]] = True |
| 11 | |
| 12 | # Compute the pairwise similarity matrix |
| 13 | normalized_embeddings = F.normalize(embeddings, p=2, dim=1) |
| 14 | sim_matrix = torch.mm(normalized_embeddings, |
| | normalized_embeddings.t( )) |
| 15 | # Extract positive and negative similarities |
| 16 | pos_sim = sim_matrix[pos_mask] |
| 17 | neg_sim = sim_matrix[~pos_mask] |
| 18 | |
| 19 | # Compute positive and negative losses |
| 20 | pos_loss = torch.mean(torch.abs(margin − pos_sim)) # Ensure |
| | positive loss for positive pairs |
| 21 | neg_loss = torch.mean(torch.abs(neg_sim)) # Ensure negative |
| | loss for negative pairs |
| 22 | |
| 23 | # Compute the regularization term: penalize drastic changes from |
| | initial embeddings |
| 24 | # The norm of the difference between the current and initial |
| | embeddings |
| 25 | normalized_initial_embeddings = |
| | F.normalize(initial_embeddings, p=2, dim=1) |
| 26 | reg_loss = torch.mean(torch.abs(margin− |
| | torch.mm(normalized_embeddings, |
| | normalized_initial_embeddings.t( )))) |
| 27 | |
| 28 | # Combine the losses to get a scalar loss value |
| 29 | loss = pos_loss + neg_loss + lambda_reg*reg_loss |
| 30 | |
| 31 | return loss |

In lines 5-10, the loss function constructs a positive mask to identify pairs of nodes connected by an edge. In lines 12-14, the loss function computes a similarity matrix for the embeddings, normalized to use cosine similarity. In lines 15-17, the loss function extracts positive and negative similarities from the similarity matrix. In line 20, the loss function calculates positive loss as the mean of absolute differences between the current similarities of connected pairs and the margin. In line 21, the loss function calculates negative loss as the mean of absolute values of the similarities for unconnected pairs. In lines 23-26, the loss function calculates a regularization loss that measures how far the current embeddings are from their initial values. In lines 28-29, the loss function calculates the total loss as the sum of positive loss, negative loss, and the regularization term, scaled by lambda. In various embodiments, the facility uses a variety of other loss functions to achieve the same or similar objectives.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system for constructing a set of vectors for text in a specialized domain, the method comprising:
accessing a first set of vectors each representing a meaning for an identified text term, all of the vectors of the first set being defined within a common embedding space;
accessing a term list for the specialized domain;
defining a second set of vectors that includes only the vectors of the first set whose identified term matches a term in the term list;
normalizing the vectors of the second set of vectors to obtain a third set of vectors;
constructing a graph that comprises a node for each of the vectors of the third set;
accessing a plurality of term characterizations, each term characterization specifying a term relevant to the specialized domain and identifying one or more other terms to which the specified term is related;
for each term characterization of the plurality:
adding a node to the graph for the term specified by the term characterization;
for each of at least a portion of the additional terms identified by the term characterization:
establishing an edge connecting the node added to the graph for the term specified by the term characterization to an existing node in the graph whose vector represents a meaning for the additional term; and
applying a graph learning technique to establish, for each of at least a portion of the added nodes, a vector representing a meaning for the term of the added node within the common embedding space.

2. The method of claim 1 wherein the specialized domain is medicine.

3. The method of claim 2 wherein term characterizations among the accessed plurality of term characterizations comprise one or more of:
medical diagnosis codes;
medical procedure codes;
prescription codes; or
lab result codes.

4. The method of claim 1 wherein the graph learning technique uses a loss function in which a positive loss value promotes the similarity of vectors for nodes that are directly connected in the graph, a negative loss value punishes the similarity of vectors for nodes that are not directly connected in the graph, and a regularization loss value that limits a degree to which vectors can be changed in a single round of learning.

5. The method of claim 1, further comprising:
accessing a document belonging to the specialized domain; and
transforming the accessed document into a sequence of vectors that correspond in the graph to terms of the accessed document.

6. The method of claim 5, further comprising:
using the sequence of vectors to train a deep learning model to make inferences based upon documents belonging to the specialized domain.

7. The method of claim 5, further comprising:
applying a trained deep learning model to the sequence of vectors to make an inference with respect to the accessed document.

8. The method of claim 7 wherein the specialized domain is medicine, and wherein the accessed document is a record in an electronic medical record system for a distinguished patient,
and wherein the inference made by applying the trained deep learning model is a medical prediction about the distinguished patient.

9. One or more memories collectively containing a vector graph data structure for a distinguished specialized domain, the data structure comprising:
a plurality of first nodes, each of the first nodes identifying a term relevant to the distinguished specialized domain and specifying a vector representing a meaning of the identified term;
a plurality of second nodes, each of the second nodes identifying a term relevant to the distinguished specialized domain and not initially specifying any vector representing a meaning of the identified term; and
for each of the plurality of second nodes, one or more edges each connecting the second node to one of the first nodes,
such the contents of the data structure are usable to perform graph learning to establish, for each of the second nodes, a vector representing a meaning of the term identified by the second node.

10. The one or more memories of claim 9 wherein each of the plurality of second nodes specifies a vector representing a meaning of the term identified by the second node, the specified vector having been established by applying graph learning to the graph.

11. The one or more memories of claim 9 wherein the distinguished specialized domain is medicine.

12. One or more memories collectively having contents configured to cause a computing system to perform a method for constructing a set of vectors for text in a specialized domain, the method comprising:
accessing a set of vectors each representing a meaning for an identified text term relevant to the specialized domain, all of the vectors of the first set being defined within a common embedding space;
constructing a graph that comprises a node for each of the vectors of the set;
accessing a plurality of term characterizations, each term characterization specifying a term relevant to the specialized domain and identifying one or more other terms to which the specified term is related;
for each term characterization of the plurality:
adding a node to the graph for the term specified by the term characterization;
for each of at least a portion of the additional terms identified by the term characterization:
establishing an edge connecting the node added to the graph for the term specified by the term characterization to an existing node in the graph whose vector represents a meaning for the additional term; and
applying a graph learning technique to establish, for each of at least a portion of the added nodes, a vector representing a meaning for the term of the added node within the common embedding space.

13. The one or more memories of claim 12 wherein the specialized domain is medicine.

14. The one or more memories of claim 13 wherein term characterizations among the accessed plurality of term characterizations comprise one or more of:
medical diagnosis codes;
medical procedure codes;
prescription codes; or
lab result codes.

15. The method of claim 1 wherein the graph learning technique uses a loss function in which a positive loss value promotes the similarity of vectors for nodes that are directly connected in the graph, a negative loss value punishes the similarity of vectors for nodes that are not directly connected in the graph, and a regularization loss value that limits a degree to which vectors can be changed in a single round of learning.

16. The one or more memories of claim 12, the method further comprising:
accessing a document belonging to the specialized domain; and
transforming the accessed document into a sequence of vectors that correspond in the graph to terms of the accessed document.

17. The one or more memories of claim 16, the method further comprising:
using the sequence of vectors to train a deep learning model to make inferences based upon documents belonging to the specialized domain.

18. The one or more memories of claim 16, the method further comprising:
applying a trained deep learning model to the sequence of vectors to make an inference with respect to the accessed document.

19. The one or more memories of claim 18 wherein the specialized domain is medicine,
and wherein the accessed document is a record in an electronic medical record system for a distinguished patient,
and wherein the inference made by applying the trained deep learning model is a medical prediction about the distinguished patient.
